(19) United States
(12) Patent Application Publication
(10) Pub. No.: US 2023/0089801 A1
(43) Pub. Date: Mar. 23, 2023

(54) DEVICE AND METHOD FOR KNOTTING A STRING END

(71) Applicant: Ruggli Projects AG, Hagendorn (CH)

(72) Inventor: Samuel SCHULER, Basel (CH)

(73) Assignee: Ruggli Projects AG, Hagendorn (CH)

(21) Appl. No.: 17/909,033

(22) PCT Filed: Mar. 5, 2021
(86) PCT No.: PCT/EP2021/055636

§ 371 Date: Sep. 2, 2022

(30) Foreign Application Priority Data
Mar. 5, 2020 (CH) 00263/20

(51) Int. Cl. A61F 13/20; A61F 13/15

(52) U.S. Cl. CPC A61F 13/2085; A61F 13/15756; A61F 2013/15821

(57) ABSTRACT

The present invention relates to a device for knotting a thread end so that a loop is formed. The device 1 comprises a knotter assembly 3 for knotting two thread sections of the thread end, as well as a driver 5 for feeding the thread sections to the knotter assembly 3. It further comprises an orbital path 4 along which the driver and the knotter assembly 3 are guided. The orbital path has a knotting region and a turnover region and is designed to guide the driver at different speeds in the knotting region and turnover region. The present invention further relates to a method for knotting a thread end, and to a device for producing tampons having a proximal retrieval thread.

The present invention relates to a device for knotting a thread end so that a loop is formed. In particular, it relates to a device for knotting a thread end of a tampon retrieval thread. The invention further relates to a method for knotting a thread end, and to a device for producing tampons having a proximal retrieval thread comprising two thread ends linked together, all respectively according to the preamble of the independent patent claims.

### TECHNOLOGICAL BACKGROUND

Tampons are used for monthly feminine hygiene and are usually rolled up or folded sheets of absorbent materials that are compressed. Rolled tampons usually comprise one or more layers of absorbent material, plus any additional non-woven layers, and are rolled up lengthwise from one end. Modern manufacturing methods additionally provide for the formation of a tapered distal head end. Folded tampons, so-called “teabag tampons”, are folded in a zigzag similar to an accordion and, if necessary, also provided with a tapered head end in a forming step like the rolled-up tampons. Regardless of the manufacturing method, all tampons require a means of extraction in order to be removed from the body orifice after use.

It is particularly important that the extraction means must not tear off under any circumstances, or that the attachment of the extraction means to the tampon, i.e. to the absorbent material or laminate, must not tear off. For this reason, tapes or threads are used which are placed around such rolled-up or folded tampons transversely to the longitudinal direction of the tape, so that the extraction means forms a loop transversely to the longitudinal extension of the tape. After the tampon has been rolled up or folded, the loop preferably comes to rest completely inside the tampon, and tearing of the extraction means from the absorbent material is practically impossible, since the extraction force not only acts on a proximal end of the tampon, but the distal end is also pulled out by the loop. As a rule, the manufacturing processes for such tampons provide for a retrieval thread to be placed around the ribbon-shaped material before the tampon is wrapped or folded and also knotted in advance. WO 2016/207242 (Ruggli Projects AG, Hagendorn-CH) shows a manufacturing process for a tampon, in which in particular an additional problem of wrapped tampons is solved. By folding the loop around the ribbon-shaped material, the extraction force acts at the distal end, i.e. at the head end of the tampon, when the retrieval thread is operated, but it may happen that the rolled-up tampon is extended and pulled out telescopically in the process. This document shows how such telescoping is substantially prevented by means of a laminate strip.

However, the latest generation of tampon manufacturing machines operate at much higher volumes than the previous ones. Swiss patent application No. 00426/18 (Ruggli Projects AG, Hagendorn-CH) shows a device for forming tampons from ribbon-shaped material, in which a retrieval thread is placed around a strip of ribbon-shaped material and knotted before winding, which is described in this publication. The device shown therein is capable of processing more than 140 tampons per minute. Existing knotting machines can no longer cope with these quantities.

There is therefore a need for knotting machines that are capable of producing high numbers of knots per unit of time without sacrificing quality.

### DESCRIPTION OF THE INVENTION

It is thus an object of the present invention to improve existing knotting devices and make them suitable for machining higher numbers of pieces. In particular, it is an object of the present invention to provide a device for knotting thread ends which is efficient and requires less maintenance.

It is another particular object of the present invention to provide a device for knotting thread ends which solves at least one problem of the known.

This object was solved with a device for knotting a thread end so that a loop is formed, as well as with a corresponding method and a device for manufacturing tampons with a proximal retrieval thread, each according to the characterizing part of the independent claims. One aspect of the present invention relates to a device for knotting a thread end so that a loop is formed. Preferably, this is the thread end of a retrieval thread for tampons.

The device includes a knotter assembly for knotting two thread sections of the thread end. It further comprises a driver, for feeding the thread sections to the knotter assembly. The device according to the invention comprises an orbital path along which the driver is guided around the knotter assembly. The orbital path has an acceleration region in which the driver can be accelerated to a first velocity. The orbital path further has a deceleration region in which the driver can be decelerated to a second speed.

Although the present invention is described above in the context of tampons for use in monthly feminine hygiene, it will be understood by a person skilled in the art that all types of tampons having a retrieval thread may benefit from the teachings of the present invention, such as surgical tampons for closing blood vessels, wounds, and anal tampons used primarily in a medical setting.

In a particular embodiment, the driver is designed to guide a thread end along the orbital path and, in particular, to unwind it from a thread bobbin. For this purpose, the driver can, for example, be equipped with a gripper and/or a clamp which is capable of gripping a section of the thread end. As an alternative to a gripper, the driver can be provided with a bobbin or roller over which the thread end is guided.

In the sense of the present invention, the driver can be regarded as deceleratable, or acceleratable, if it is operatively connected on its path, e.g., with a drive means which is deceleratable and/or acceleratable.

In a particular embodiment, the driver may engage, e.g. by means of a pin, with a driven chain which is mounted on the orbital path. This chain can be accelerated to a speed by means of a drive and decelerated if necessary. The driver engaging in the chain would also be accelerated or decelerated.

In an alternative embodiment, the driver could be rigidly attached to a movable carriage mounted on the orbital path, which in turn is accelerated, or decelerated, along the orbital path so that the acceleration and deceleration action affects the driver.

In a further alternative embodiment, the driver can be driven by a belt, which can be accelerated or decelerated along the orbital path. Also conceivable would be a magnetic orbital drive in which excitable magnets along the orbital path are capable of accelerating or decelerating a driver mounted on an orbital path.

In a particular embodiment, the orbital path includes an annular bearing and is itself mounted for rotation about a central unit. The driver is then, for example, rigidly connected to the rotatably mounted orbital path. The orbital path can be driven, e.g. by providing a toothing on the orbital path, which can be driven and accelerated or decelerated by means of a drive, e.g. via a toothed belt.

In a particular embodiment, the driver may be directly driven. Preferably, the device according to the invention features a direct drive for the driver. In a further particular embodiment, this direct drive can be designed as an electric direct drive, in particular as a torque drive. For this purpose, for example, a stator can be provided on the outside or inside of the orbital path and the driver as a rotor.

In accordance with the present invention, a drive control system may be provided which is adapted to define an acceleration region and a deceleration region for the orbital path. The drive control system could be designed to ensure that the driver traverses the orbital path in one revolution at at least two different speeds, where, for example, the different speeds are initiated by a deceleration region and an acceleration region, respectively. In another particular embodiment, the drive control system is configured to provide a first speed that is maintained in the knotting region.

In a particularly preferred embodiment, the drive control is designed to provide a second speed of the driver which is maintained in a turnover region.

For the purposes of the present invention, the turnover region may be understood as an area in which one or more thread sections of thread carried by the driver are laid over a web feed area so that they wrap around a corresponding length of web of a ribbon-shaped substrate to be provided with a retrieval thread. Analogously, in the sense of the present invention, the knotting region may be regarded as a further region in which at least two thread sections of the thread end are guided to the knotter assembly in such a way that the knotter assembly knots these two together.

In operation, this would then mean that an eye, a loop, or a loop of a retrieval thread end is formed in the turnover region, and the resulting free thread ends are linked in the knotting region.

Knotter assemblies suitable for carrying out the present invention are known. In their simplest embodiment, the knotter assemblies suitable for the device according to the invention comprise at least one knotting mandrel. The knotting mandrel is rotatably mounted and driven within a knotting ring. In general, the knotting mandrel rotates in a direction of rotation that is opposite to the direction of rotation of the driver on the orbital path. The knotting mandrel engages in the thread sections approached by the driver and performs a rotation during the time the driver is traveling in the knotting region. The rotation of the knotting mandrel then creates a knot.

In a particular embodiment, the knotter assembly has an aperture which is suitable for guiding the two thread sections into the effective region of the knotter assembly when they are guided into the effective region of the knotter assembly by the radially passing driver. In a particular embodiment, the device is adapted to form a knot, that is, to knot a thread end, during one revolution of the driver on the orbital path.

In a particular embodiment, the driver is oriented to be guided radially on a circular orbital path. In an alternative or complementary embodiment, the driver is rigidly connected to the orbital path and the orbital path is adapted to be radially guided.

In a particular embodiment, the device further comprises a thread feed for continuously feeding a thread end to the driver.

In a particularly preferred embodiment, the thread feed comprises a thread bobbin with a wound-up thread, and in particular at least one unwinding reel via which the thread is connected to the driver. As the driver moves, the thread is unwound from the thread bobbin and fed once around the entire device, essentially forming a loop of two pieces of thread.

For the purposes of the present invention, a thread end may always be considered to be the machined end portion as viewed with respect to the total length of the thread on the bobbin.

The pieces of thread, as they may be understood in the sense of the present invention, form the two legs of a loop formed substantially by an enveloping movement of the driver, forming an eye in which the ribbon-shaped substrate will later be located. This loop is typically placed around the ribbon-shaped substrate transversely to the longitudinal direction of the ribbon-shaped substrate, so that pulling on the knotted end of the loop exerts force transversely to the longitudinal direction of the ribbon-shaped substrate.

For the purposes of the present invention, the side of the loop opposite the knot may also be referred to as the distal end of the retrieval thread, while the knot may be referred to as the proximal end of the retrieval thread. This would correspond to the usual terminology when considering a tampon, with the distal end usually formed by a conically pointed shaped tampon head.

In a particular embodiment, the knotter assembly is configured to form a knot by rotating the knotting mandrel about an axis of rotation in a direction opposite to the direction of rotation of the driver. In particular, the knotting mandrel rotates more than one complete revolution about its axis of rotation when forming the knot. Preferably, the knotting mandrel is designed to complete between 1 and 1.8 revolutions when forming the knot, in particular between 1.1 and 1.6 revolutions, most preferably about 1.5 revolutions.

In a particular embodiment, the knotting mandrel is driven to make more than one complete revolution in the direction opposite to the direction of rotation of the driver during the period of time that the driver moves through the knotting region. In operation, this can mean that the knotting mandrel completes between 1 and 2 revolutions while the driver travels around a knotting region and returns to a starting position, in particular by completing the started revolution while the driver travels around the turnover region.

In a particular embodiment, the device further comprises a cutting element for severing the knotted loop of the thread end. Particularly preferably, the cutting element acts on the piece of thread that is still connected to the bobbin at the proximal end of the loop. Particularly preferably, the cutting element is mounted in such a way that the cutting is performed by the movement of the driver, i.e. the driver guides the piece of thread to be cut past the cutting element in such a way that it is cut. In its simplest embodiment, the cutting element may be a blade mounted in the direction of travel of the driver, through which the driver guides the thread against a resistance so that the thread is cut by the cutting element.

In a particular embodiment, the cutting element is curved. In a particular embodiment, the interface of the cutting element now forms the new end piece over which the driver unwinds further thread in the further orbital path and a new thread end is formed to initiate a new cycle.

In a particular embodiment, the orbital path is substantially circular. Particularly preferably, the orbital path is circular. By unwinding the thread with a circular movement of the driver, the overall design can be made more compact and an accessibility of the individual component can be improved. In addition, the wear of the components is lower.

In this embodiment, the knotting region, the acceleration region, the turnover region, the deceleration region may be defined as angles of the circular orbital path. The angles can follow the mentioned sequence and overlap. In another particular embodiment, the device is configured such that the driver completes a cycle with one revolution of the orbital path about its axis of rotation, particularly about the center of a circular orbital path.

In a particular embodiment, the cycle comprises two speeds in which the driver is guided along the circular orbital path. A first speed in the knotting region is designed to be lower than a second speed in the turnover region. Particularly preferably, the first and second speeds are matched to the rotational speed of the knotting mandrel. The first speed is such that the knotting mandrel is able to complete between 1 and 2 times a revolution about an axis of rotation. The second speed is such that the knotting mandrel is able to complete the remaining revolution, i.e. the difference between a started and finished second revolution. For example, it can be ensured that the knotting mandrel is back in its initial state and ready when the driver re-enters the knotting region. Alternatively, the knotting mandrel can also complete more than the remaining revolution, i.e. the difference between a started and completed second revolution, i.e. additionally complete N whole revolutions. Where a complete revolution represents a complete rotation around the axis of rotation, and defines an angle of 360°.

In a particular embodiment, the device according to the invention comprises a path inlet for passing a ribbon-shaped substrate through the effective region of the driver.

In a particular embodiment, this path inlet is a recess through which the ribbon-shaped substrate can be conveyed into the effective region of the driver. For the purposes of the present invention, the effective region of the driver is defined by its radial movement along the orbital path. In other words, the effective region of the driver is given as soon as an object is within the orbital path of the driver. In practice, this may mean that the driver unwinds a thread end and carries it along the orbital path in such a way that it wraps it around a ribbon-shaped substrate located in the path inlet.

Accordingly, in a particular embodiment, the path inlet is formed laterally to the knotter assembly. Particularly preferably, the thread end is placed around the ribbon-shaped substrate during an orbital path of the driver on the orbital path at the second speed.

In a particular embodiment, the device includes a holding device to hold a loop formed by the circulation of the driver on the orbital path. Particularly preferably, the holding device is formed laterally to the knotter assembly. Most preferably, the holding device is formed laterally to the path inlet so that the loop is held in such a way that a ribbon-shaped substrate comes to rest within the legs of the loop.

In a particular embodiment, the holding device is designed as a latch. The latch is designed to exert a restoring force on a loop end of the thread end to be knotted. In particular, the latch is designed to exert a releasable restoring force on a loop end of the thread end to be knotted.

In a particular embodiment, the holding device is driven. This can be achieved, for example, by providing the holding device with an activatable pneumatic cylinder.

In an alternative or complementary embodiment, the holding device is provided with a restoring force exerting component such that the holding device maintains tension on the retained loop with respect to the driver and the knotter assembly. In the variant in which the holding device is designed as a latch, it can also be equipped with a mechanical end stop that locks the extended latch. When the latch is released, it can release a loop and enter a released state. Preferably, the latch is then equipped so that it can be automatically moved to its original position.

In a particular embodiment, the latch comprises a pneumatic cylinder.

In an alternative or supplemented embodiment, the latch comprises a spring.

In a particular embodiment, the holding device is configured to be adjustable with respect to a path inlet so that different loop lengths can be accommodated. For example, depending on the selected distance of the holding device to the path inlet or to a knotter assembly, correspondingly longer or shorter thread sections can be used to form the loop.

In a particular embodiment, the holding device comprises at least two holding teeth. The holding teeth may be configured such that one end of a thread section is held by a holding tooth in each case, so that the holding device holds up a corresponding eye of a loop in which, for example, a path inlet may be guided in order to guide the ribbon-shaped substrate through.

In a particular embodiment, the latch is configured to exert a holding force on the loop as long as the loop has a mating pull. In operation, this can mean that once a knot has been tied and the end of the thread has been severed, there is no longer any counter-tension, as a pulling force is no longer exerted on the loop by the driver. The latch may be configured so that lack of a counter pull releases the loop. This release of the loop can cause it to be pulled along by a conveyed ribbon-shaped substrate around which it is placed.

In a particular embodiment, the device comprises a guide plate for guiding the ribbon-shaped substrate through the effective region of the driver. This guide plate can, for example, be arranged at right angles to the path inlet.

In a particular embodiment, the guide plate is designed in such a way that, after a holding device has been released, a formed loop is first folded over onto the guide plate and is then passed on to the next processing step together with the corresponding web substrate section by conveying the ribbon-shaped substrate.

In a particular embodiment, the guide plate has a tapered end face in the longitudinal direction of the ribbon-shaped substrate.

In a particular embodiment, the width of the guide plate is aligned with the length of the retrieval thread to be achieved.

In a particular embodiment, the device according to the invention further comprises a knot control for checking the knot tied by the knotter assembly. In particular, the knot control may include optical, mechanical, and/or electrical knot control. For example, visual knot control can be achieved by having a camera guided into the knotter area and visually inspecting the knots. A mechanical knot control can be provided, which prevents the knotted retrieval thread from being conveyed further if there is no knob.

In a particular embodiment, the orbital path is drivable. Particularly preferably, the orbital path has toothing through which it can be driven by means of a toothed belt. Particularly preferably, it can be accelerated uniformly and performs a rotation about an axis of rotation. In this embodiment, the drivers may be rigidly connected to the orbital path by being riveted and/or bolted to it.

Particularly preferably, the drivers are designed to be interchangeable and can be detachably attached to the drivable orbital path. In this embodiment, a toothed belt can be connected to a belt drive which, via a drive control, is capable of accelerating or decelerating the orbital path in a region-segmented manner according to the invention. This can result in the orbital path describing a radius in which different speeds of the driver are provided.

In a particular embodiment, the drive control system is designed to provide a continuously acceleratable circulating drive.

In a particular embodiment, the knotter assembly includes at least one knotting mandrel rotatably driven parallel to the direction of rotation of the orbital path. The present invention provides a device for knotting a thread end so that a loop is formed, which is easy to maintain and is adapted to supply ribbon-shaped substrates with the corresponding loops at a high frequency so that they can be formed into retrieval threads of tampons. The device according to the invention accomplishes this in a continuously operating manner and is thus adapted to the requirements of modern continuous manufacturing processes. For a person skilled in the art, it is self-evident that all the above-mentioned embodiments can be combined with one another in an embodiment according to the invention, provided that they are not mutually exclusive.

Another aspect of the present invention is a method for knotting a thread end, in particular two thread sections of a thread end, so that a loop is formed which forms a retrieval thread for tampons.

In a particular embodiment, this method is carried out with a device according to the invention as described above. Accordingly, the functional features described above with respect to the device are at the same time possible method features of the method according to the invention.

The method according to the invention comprises the step of guiding a thread end of a thread by means of a driver movable on an orbital path along the orbital path around a knotter assembly and an envelope element, so that a loop of two thread sections of the thread end is formed between the knotter assembly and the envelope element.

The method according to the invention further comprises the step of knotting the two thread sections by a knotter assembly and releasing the loop at the envelope element. In the method according to the invention, the orbital path has a knotting region, which is traversed by the driver at a first speed, and a turnover region, which is traversed by the driver at a second speed. For this purpose, an acceleration region, in which the driver is accelerated to a second speed, and a deceleration region, in which the driver is decelerated to a first speed, can be provided between the knotting region and the turnover region at the transitions.

In a particular embodiment, the ratio of the first speed to the second speed is between two to one and six to one.

In operation, for example, a driver would guide a thread end from a thread bobbin along the orbital path. This orbital path guides the thread past the envelope element so that tension is created between the envelope element and the driver. Further, this creates an eye of a loop. On its further path, the driver now guides two thread sections to the knotter assembly, and this knots the two thread ends.

In a particular embodiment of the method according to the invention, a feeding of a thread end from a thread on a bobbin to the driver movable over the orbital path occurs via a restoring force. This can result in a steady tension being maintained on the thread, regardless of the trajectory the driver completes on the orbital path. It should be noted that although the driver can follow a circular orbital path, the thread can be guided directly by the thread package or by a deflection roller that may be connected in between.

A restoring force can act on the thread by, for example, a spring-loaded lever or a driven intermediate bobbin being arranged between the driver and the thread bobbin. In a particular embodiment, these elements are located outside the radius of the orbital path.

In a particular embodiment, the releasing of the loop comprises cutting the end of the thread from the thread. This cutting can take place essentially at the same time as the knotting of the thread sections. In particular, this cutting takes place shortly after the knotting of the thread sections.

In a particularly preferred embodiment, cutting off the end of the thread also heralds the releasing of the loop by preventing it from exerting a restoring force on the knotted loop due to a lack of counter-tension, thus releasing it.

In a particular embodiment, the ratio of the first speed to the second speed is between one to two and one to six. In other words, the movement of the driver in the range of the first speed occurs at a speed that is substantially between one-half to six times less than the speed in a second range.

In another particular embodiment, the first speed is the speed that the driver has when it passes through a knotting region, that is, passes through a region in which the thread sections are at the knotter assembly and knotting of the thread sections by the knotting mandrel takes place.

In a particularly preferred embodiment, the knotting region, the turnover region, and the acceleration or deceleration region are defined as angles of a circular orbital path. In a particular embodiment, the knotting region comprises a range of between 5 and 45° of the orbital path, preferably between 10 and 30° of the orbital path, more preferably 15° of the orbital path. Accordingly, the remainder of a complete orbital path, that is, the remainder between 355 and 315°, in particular between 350 and 330°, further in particular 345° forms the turnover region.

Particularly preferably, the orbital path has an acceleration region and a deceleration region in which the corresponding speeds are recorded. Particularly preferably, the acceleration region and the deceleration region are located in the turnover region.

In these regions, the driver is accelerated or decelerated to the corresponding speed it travels in the turnover region, respectively in the knotting region. Particularly preferably, the acceleration and deceleration of the driver is performed by a drive and is controlled by a drive control system. In this example, a drive control system could be designed to intermittently control a motion of the driver that transitions from a first speed to a second speed and back again, in a cycle so that the same angular ranges of an orbital path on which the driver is moving are always used for the corresponding acceleration or deceleration.

In a particular embodiment of the method according to the invention, a position start position P₀ may be considered, for example, a position in which the driver makes an angle to the center of a circular orbital path that is 0° or substantially 0°. In the context of the present invention, substantially 0°, or substantially X°, is to be understood as a deviation of at most 1°.

In a particular embodiment, the knotting region includes an angle of between 0 and 45°, in particular of between 0 and 30°, in particular of 15°.

In another particular embodiment, an acceleration region adjoins a knotting region. In another particular embodiment, the knotting region is at an angle of between 0° and 15° and the acceleration region begins directly after it.

In a particular embodiment, releasing the loop on the envelope element provides for releasing a resettable latch. The latch is configured to exert a releasable restoring force in the direction of the driver and/or knotter assembly.

The latch is preferably designed to be releasable and automatically returnable to its original state. This can be done, for example, by means of a pneumatic cylinder or also by means of a spring provided with a set restoring force. It would also be conceivable to use a magnetic control system to transfer the latch from a released position to a position in which it exerts a restoring force.

In a particular embodiment, the method according to the invention further comprises the steps of providing a device for knotting a thread end as described above, and passing a ribbon-shaped substrate through the effective region of the driver so that the thread end is laid around the ribbon-shaped substrate. In particular, the thread end is laid around the ribbon-shaped substrate such that when the driver orbits the orbital path, the thread end is laid around the ribbon-shaped substrate, wherein the passing of the ribbon-shaped substrate is arranged substantially normal to an orbital path of a driver.

In operation, a device according to the invention can be installed in a system for the production of tampons in such a way that it is brought into operative connection with the device perpendicularly to the axis of rotation of the orbital path or of the driver. For this purpose, a corresponding feed aperture can be provided, which is capable of feeding a ribbon-shaped substrate, for example, into a web feed as described above. The web feed is configured such that a formed loop is disposed around the web feed by the movement of the driver, such that a ribbon-shaped substrate is disposed inside this web feed perpendicular to, or at least substantially normal to, the longitudinal orientation of the thread. If the loop is formed and detachment takes place, the loop falls onto the ribbon-shaped substrate.

In a particular embodiment, the ribbon-shaped substrate is guided over a guide plate onto which the loop initially falls and is placed onto the ribbon-shaped substrate by a tapered end face as it progresses, without interfering with the ribbon-shaped substrate in its conveying path.

Accordingly, it is another aspect of the present invention to provide a device for producing tampons having a proximal retrieval thread. The retrieval thread comprises two thread ends linked together. The device comprises a device for knotting two ends of thread as described at the beginning.

It further comprises a conveying device for conveying ribbon-shaped substrate into an effective region of the device for knotting the two ends of the thread. In particular, the conveying device is arranged substantially normal to an orbital path of a driver for feeding two thread sections to a knotter assembly.

Another aspect and/or particular embodiment of the present invention relates to a device for knotting a thread end so that a loop is formed around a ribbon-shaped substrate, in particular a thread end of a retrieval thread for tampons. The device may further comprise any features of the above device not in conflict with the following features.

This device includes a knotter assembly for knotting two thread sections of the thread end.

It further comprises a driver for feeding the thread sections to the knotter assembly, wherein the driver is rotatably supported along an orbital path around a path inlet for passing a ribbon-shaped substrate, in particular is rotatably supported along a circular orbital path. The device further comprises a pickup disposed substantially normal to the path inlet, which is configured to receive a thread section from the driver, and deposit the thread onto the ribbon-shaped substrate. In the context of the present invention, substantially normal is to be understood as being disposed with respect to the plane of the ribbon-shaped substrate within an angular range of between 85° and 95°.

By means of a pickup arranged in such a way, the thread can be gently deposited on the ribbon-shaped substrate, for example a absorbent cotton tape. Overall, the depositing movement is calmed, which makes it possible to increase the process speed and improve reliability. The device runs more smoothly overall despite the high speed. The increased reliability also allows maintenance intervals to be shortened and malfunctions to be reduced, which also allows better utilization of the device overall.

In a particular embodiment, the pickup is substantially made of metal. This can increase durability and reduce wear. The pickup is particularly preferred to be made of stainless steel. Further preferably, the pickup is manufactured in a lightweight design at least in its moving parts, i.e. material is saved wherever possible, for example by providing recesses in a pickup lever which can lead to material and thus also weight savings.

Alternatively, the pickup, in particular at least one pickup hook for receiving a thread section, is made of a ceramic material.

In a particular embodiment, the device comprises a guide plate for guiding the ribbon-shaped substrate through the effective region of the driver. In another particular embodiment, the device comprises a stop plate. This stop plate can be positioned between the guide plate and the pickup so that the ribbon-shaped substrate is not touched by the pickup when the thread path is laid down by the pickup. This can increase the smooth running of the device.

In a particular embodiment, the pickup comprises a pickup spring that exerts a restoring force on a pickup hook configured to receive a thread section. The restoring force is preferably set to be overcome by knotting two thread sections of the thread end by the knotter assembly. In operation, for example, knotting would then result in lowering of the pickup lever against the restoring force of the pickup spring, allowing the loop thus formed to be gently deposited onto the substrate tape. As soon as the thread section is released from the pickup hook, the restoring force can act on the pickup hook in such a way that it snaps back into its original position, ready to receive a next thread section of a following thread end.

By substantially smoothly sliding a loop onto the ribbon-shaped substrate, the smoothness of the device can be increased, and a further increase in process speed can be made possible.

In a particular embodiment, the pickup configured to deposit a thread section from the driver onto the ribbon-shaped substrate includes a spring element that exerts a restoring force on the pickup. The spring element can facilitate return of the pickup to an angled state after a thread section has been laid down.

In a particular embodiment, the knotter assembly has an aperture which is suitable for guiding the two thread sections into the effective region of the knotter assembly when they are guided into the effective region of the knotter assembly by the radially passing driver.

In a particular embodiment, the knotter assembly has a knotter eyelet which is suitable for guiding at least one thread section into the knotting region of the knotter assembly when they are guided into the knotting region of the knotter assembly by the radially passing driver. Preferably, the knotter eyelet has a guide geometry for this purpose, which is designed to be aligned against the direction of rotation of the driver.

The device for manufacturing tampons according to the invention has a conveying device for conveying the ribbon-shaped substrate, which conveys into the effective region of the device for knotting two thread ends in such a way that, during an orbital movement of a driver along an orbital path, the thread end is laid around the ribbon-shaped substrate essentially transversely to the longitudinal axis of the latter.

In the following, the present invention will now be explained in more detail with reference to figures and specific examples, without being limited to them. For a person skilled in the art, further advantageous embodiments which can be realized in a solution according to the invention will result from the study of these examples and figures.

The figures are schematic, and for simplicity the same parts have been given the same reference numbers.

### FIGURE DESCRIPTION

Examples of embodiments of the invention are described with reference to the following figures. Showing:

FIG. 1 schematically the structure of a device according to the invention;

FIG. 2 schematically an area breakdown of an orbital path;

FIG. 3 an example of a device according to the invention;

FIG. 4a schematically a latch as it can be used in a device according to the invention in a clamped position;

FIG. 4b the corresponding latch of FIG. 4a in a relaxed position;

FIG. 5a another example of a device according to the invention;

FIG. 5b alternative embodiment of the device according to FIG. 5a

FIG. 6 a schematic representation of a suitable knotter assembly;

FIG. 7a a view of a knotting mandrel without eyelet;

FIG. 7b a view of a knotting mandrel with eyelet, and

FIG. 8a an alternative example of a device according to the invention;

FIG. 8b a schematic section showing a detailed view of an unstressed pickup of embodiment 8a;

FIG. 8c a schematic section showing a detailed view of a tensioned pickup of embodiment 8a;

FIG. 8d a schematic section showing a detailed view of a knotter assembly of embodiment 8a, and

FIG. 9 schematically the structure of the embodiment according to FIG. 8a.

### DESIGN OF THE INVENTION

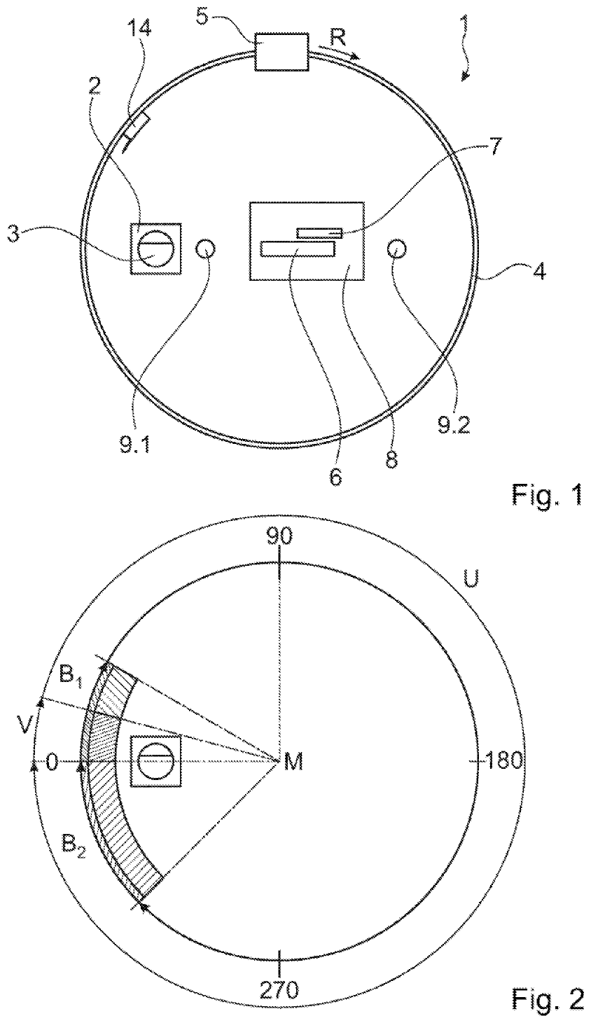

FIG. 1 shows a schematic structure of a device 1 according to the invention for knotting a thread end. The aim of knotting is that two loose ends of a thread first form an eye, and the loose ends are knotted in such a way that together they form a tight loop. In the application method described, the loop is wrapped around a ribbon-shaped material so that both legs of the loop each come to rest on one side perpendicular to the longitudinal direction of the belt. When the ribbon-shaped material is rolled up, for example to form a tampon for feminine hygiene, the loop comes to rest inside the tampon. The knotted ends of the thread protrude from the tampon and the knot and the loop formed with the knot can serve as a retrieval thread, for example, to remove the tampon.

An orbital path 4 is initially provided for the device 1 for the purpose of wrapping around a web material and knotting the ends of the thread. In the present example, the orbital path is circular. Advantageous effects can be achieved by the circular design of the orbital path. Objects moving on a circular path can be moved at a constant radial speed. Bearings and/or drive means experience a substantially uniform load, and can be designed accordingly to avoid or at least minimize imbalance effects. In addition, the control of the individual elements (see later synchronization of the knotting mandrel with the thread driver) is simplified. Acceleration and deceleration of the elements can be carried out in coordination with angular segments, so that fine adjustments are also possible—such adjustments are particularly useful when a device according to the invention is adapted to a new thread material and/or web material. For this purpose, depending on the thread tension, elasticity and frictional resistance, the person skilled in the art can adjust the control to the movement of the elements as required.

In the present example, a driver 5 is moved along this orbital path 4 at different speeds in a direction of rotation R. For simplicity, this direction of rotation R corresponds to the clockwise direction and will be taken as a reference in the further course of the application to explain the direction of rotation of other components. Of course, a device according to the invention can also be designed as a mirror image, and the corresponding directions of rotation can be exactly opposite.

The driver 5 is used to carry a thread from a thread bobbin (not shown in FIG. 1) along the orbital path 4, thereby placing it around certain elements of the device 1.

On its way, the driver 5 wraps a thread in particular around a first pin 9.2, which in the present example serves as an envelope element. The pin 9.2 keeps one eye open during continuous movement around the orbital path. When moving along the orbital path 4, the driver 5 performs a complete circle as a whole and allows the thread, which may be arranged, for example, by a thread bobbin on the opposite edge of the first pin 9.2, to form a loop between the thread bobbin and a second pin 9.1. In the simplest embodiment, this second pin 9.1 is designed as a mandrel or sword which projects into the effective space of the driver 5 from a rear wall on which the elements of the device are placed and may also be drivable. In the present simplest case, the first pin 9.1 is also a mandrel, for example a mandrel tapering towards the viewer of FIG. 1, from which a knotted thread loop can easily slide off in the direction of the viewer, for example in which the thread loop is entrained by a belt conveyor running normally to the orbital path 4 of the device 1. In particular and particularly advantageous embodiments, the first pin 9.2 may be replaced by an actuated lever, or other powered holding device, such as a releasable latch.

In order to prevent that the thread end released from the first pin, which is formed as a mandrel, hits the ribbon-shaped substrate too hard with the loop, a deflection fin 7 is provided, which also extends into the effective region and into the interior of the loop, catches it when it is released from the mandrel and gently releases such a tapering end in the tape running direction onto the ribbon-shaped material.

Ribbon-like material enters the effective region of the driver 5 through a path inlet 8. In the simplest embodiment, the path inlet 8 is a recess in the rear wall of the device. A web guide plate 6 projects into the effective region of the device through the path inlet 8 and is arranged to guide a ribbon-shaped material into the device via a drive device, for example rollers (not shown). On this tape guide plate 6, ribbon-shaped material is conveyed into the active area of the driver 5. Once the driver has passed the second guide pin 9.1, the tape guide plate 6 and thus the ribbon-shaped substrate is located within an eye of a loop formed by two legs of the thread carried by the driver 5.

In the course of this movement, the driver 5 passes through a turnover region at a certain speed.

At the opposite end of the first pin 9.1 is arranged a knotter assembly 3, which is arranged within a knotter eyelet 2.

In the present example, the driver now travels through a knotting region at a speed that is different from the speed of the turnover region. This allows the driver 5 to travel down the turnover region at a speed between twice and five times the speed it travels in the knotting region. This knotting region begins where the action of the knotter assembly starts to act on the end of the thread, respectively where the knotter assembly links the two legs of the loop and forms a knot. In the present example, the knotting region begins approximately there after the driver 5 has passed the second pin 9.1 and has made a loop around the first pin 9.2, inside which the tape guide plate 6 is present. The corresponding knotting region and turnover region can be defined as the angle of the orbital path 4.

The speed of the driver 5 is variable and the driver 5 may be controlled and driven to accelerate and decelerate. If the driver 5 is in the knotting region, a knotting mandrel (not shown in FIG. 1) rotates in the knotter assembly 3 with a rotation, in particular one and a half times, in the opposite direction to the direction of rotation R1 of the driver 5. Finally, the driver 5 passes through the effective region of a cutting blade 14, which cuts off the end of the thread, thus enabling the knotted thread loop to be led away. The driver 5 picks up another thread end and continues the movement in rotation direction R.

In FIG. 2, the area breakdown according to FIG. 1 of the effective regions using angular openings of the orbital path 4 according to an arrangement as shown in FIG. 1 is explained again in more detail. FIG. 2 schematically shows again the orbital path 4 from FIG. 1. Also shown is a center M of the circular orbital path 4. The driver is guided on a radius of the orbital path 4. A process cycle begins when the driver 5 guides a loose thread end over an turnover region U. This turnover region U may include a first acceleration region B1. In the acceleration region B1, the driver accelerates to a second speed. This Second Speed can be more of 500 rpm (relative to the orbit) and in the present example is in the range of 510 rpm. In the acceleration region B1, an acceleration of between 200 and 950 rad/s²can take place.

The initial speed of the driver 5 at the beginning of the acceleration region B1 corresponds to a constant speed which the driver maintains during the entire passage through the knotting region V. The constant and comparatively lower speed in the knotting region V enables the knotter assembly 3 to knot the loop ends fed by the driver. In the present example, the speed of the driver is five to six times lower during the knotting region V than the maximum speed in the turnover region U is. Specifically, the driver 5 here leaves the knotting region V at a speed of between 50 and 15, in particular of about 90 rpm.

In order to ensure that, when the knotting region V is re-entered, the speed of the driver is decelerated to the constant speed, which is between half and six times lower than the maximum speed travelled by the driver in the turnover region U, the turnover region U comprises a second acceleration region B2, de facto a deceleration region B2, in which the speed around the driver is decelerated, for example with an acceleration between 200 and 950 wheel/s².

In the present example, the knotting region may include an angle of substantially 15°. Thus, depending on the setting of the parameters by the person skilled in the art, there is a time interval in the knotting region of between 0.015 and 0.05 seconds for the formation of the knot. Overall, the speed of the driver in this specific exemplary embodiment is such that it performs a complete cycle within a period of between 0.08 and 0.18 seconds. In the present example, a time period of 0.028 seconds could be defined for the knotting region V, and a time period of 0.053 seconds for the turnover region U at an acceleration in the acceleration regions B1, B2 of +, respectively − approx. 940 rad/s².

This has made it possible to provide a knotting device as described at the beginning of this article, which is capable of very high throughput speeds, and at the same time can run with low material wear and low maintenance.

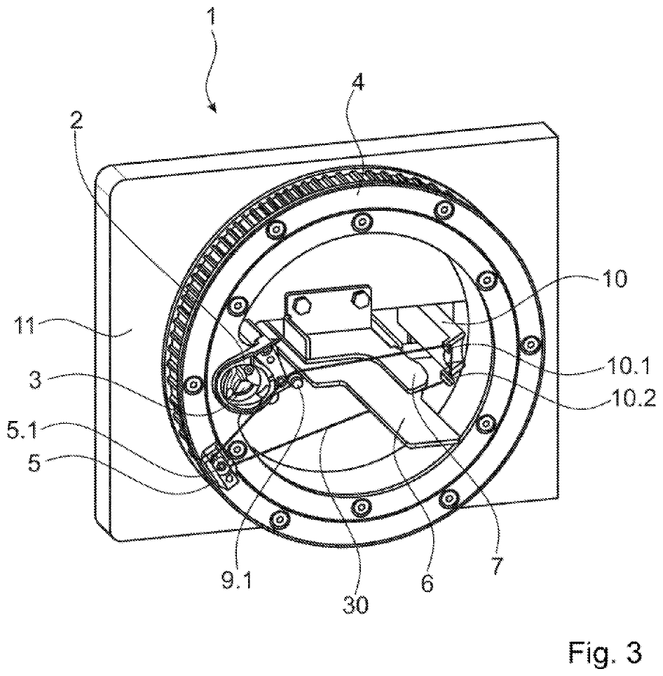

FIG. 3 shows a possible embodiment of a device 1 according to the invention. In this example, a driver 5 is rigidly connected to an orbital path 4, which can be driven by means of toothing via a toothed belt (not shown). The orbital path 4 is arranged around a ring bearing. The driver 5 has a thread clamp 5.1 which grips a thread end 30. By its movement on the orbital path 4, the driver 5 unwinds thread from a (not shown) thread bobbin. In the present example, the uncoiling occurs via the movement of the driver 5 on the orbital path 4, and is thus continuous. Also conceivable, however, are intermediate deflection rollers or an unwinding roller which actively feeds the thread to the driver.

In the present example, the first pin 9.2 is replaced by a holding device 10, which has holding teeth 10.1, 10.2. These holding teeth 10.1, 10.2 hold open a loop end of the thread end 30 so that an eye is formed. In the actual operation of tampon manufacture, this forms the end face, i.e. the distal of a retrieval thread, which essentially comes to lie inside the tampon.

As the driver 5 moves along the orbital path 4, the legs are placed on the holding teeth 10.1, 10.2 in such a way that the loop is held up until knotting of the proximal loop ends, i.e. the thread ends facing away from the end face, has taken place. Inside the stopped loop is a web guide plate 6, on which a ribbon-shaped substrate is guided through the effective area of the device 1. A deflection fin 7 is provided to initially catch any loosening of the thread and to allow it to settle onto the ribbon-shaped substrate via a tapered end. The deflection fin 7 is initially designed so that it extends parallel to the conveying direction of the ribbon-shaped material into the effective area of the device and tapers on the end face to allow the loop to slide off smoothly. In particular embodiments, the deflection fin 7 can be installed in the device 1 in a replaceable and/or adjustable manner to facilitate fine adjustment of the operating parameters to the thread length, elasticity, etc. for the person skilled in the art.

The driver 5 guides the loop end in its movement around the holding teeth, around the tape guide plate 6 and around the deflection fin 7 to a knotter assembly 3. There, both loop ends are guided and knotted by a knot eye at a knotting mandrel.

Downstream of the knotter assembly, a blade may be provided to sever the end of the thread (not shown).

The entire device 1 is attached to a machine frame 11.

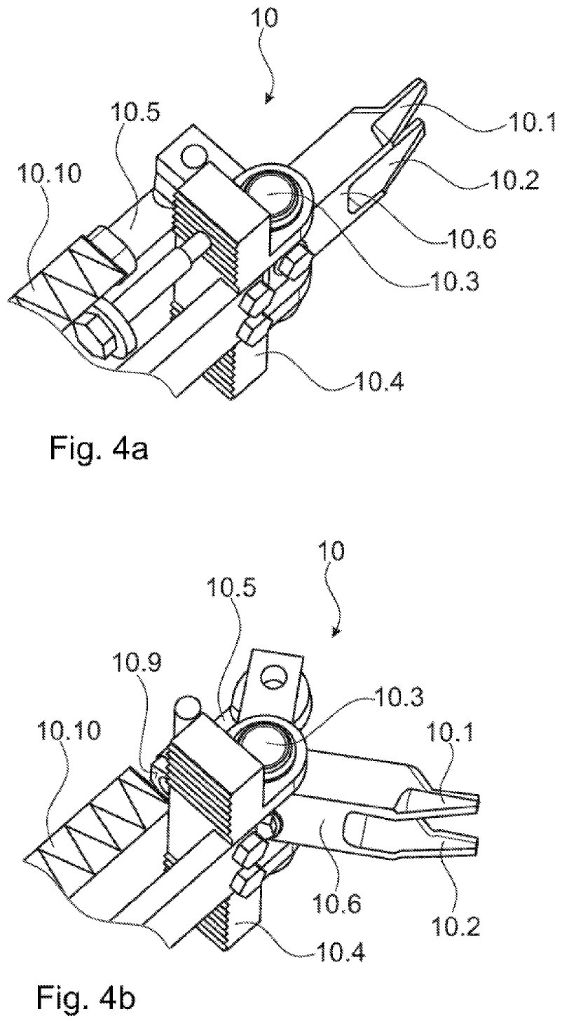

A holding device 10 which can be used for the device according to the invention as shown in FIG. 3 and which is designed as a latch is shown in FIGS. 4a and 4b. The holding device 10 has two holding teeth 10.1, 10.2. These serve to hold open a loop which is formed by the driver 5 during its radial movement on the orbital path around the holding teeth 10.1, 10.2. As a latch, the holding teeth 10.1, 10.2 are formed on a latch arm 1.6 and are rotatably mounted around a latch pin 10.3.

The holding device 10 is also designed with a locking screw and a locking plate 10.4 and can thus be mounted in the device in a displaceable manner. Once again, this makes it easier for the person skilled in the art to adjust the device to the properties of the thread material and a desired tape width, speed, thread section, etc. For this purpose, the locking plate 10.4 can also be arranged to be displaceable along a groove in an axis to the web feed via a screw. This displaceability guarantees in particular that different thread sections and desired loop sizes can be accommodated.

To ensure that the latch arm 10.6 returns to its original starting position after being released once, a restoring force or mechanism can be provided, such as a spring or pneumatic cylinder.

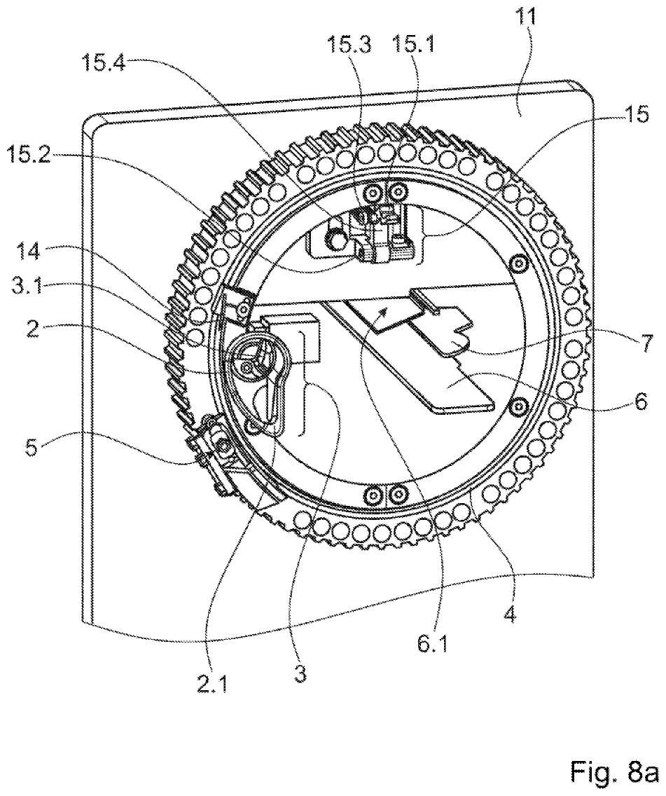

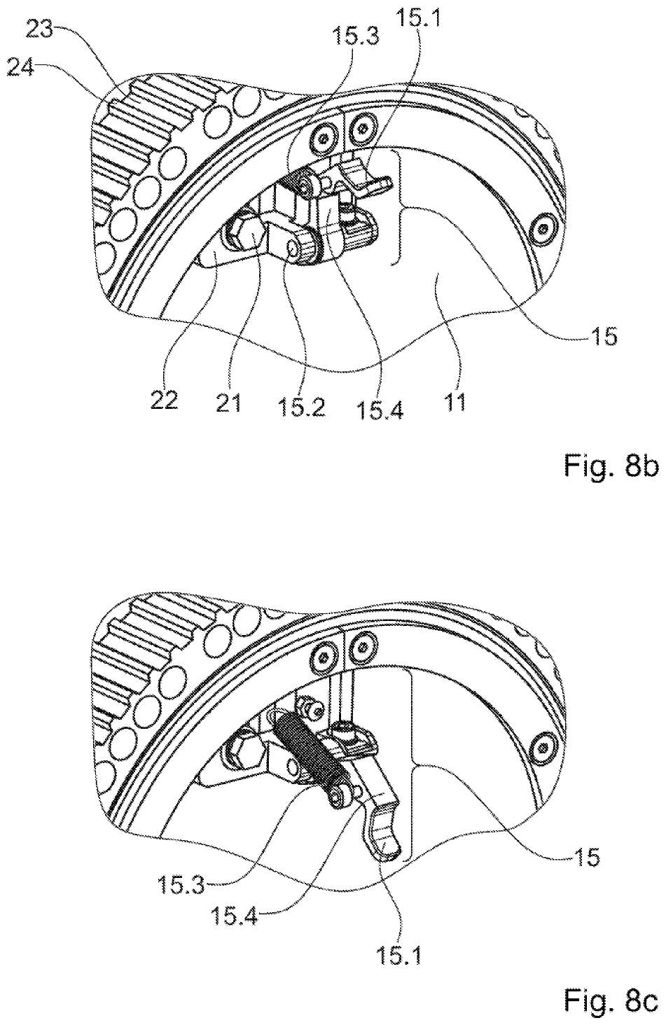

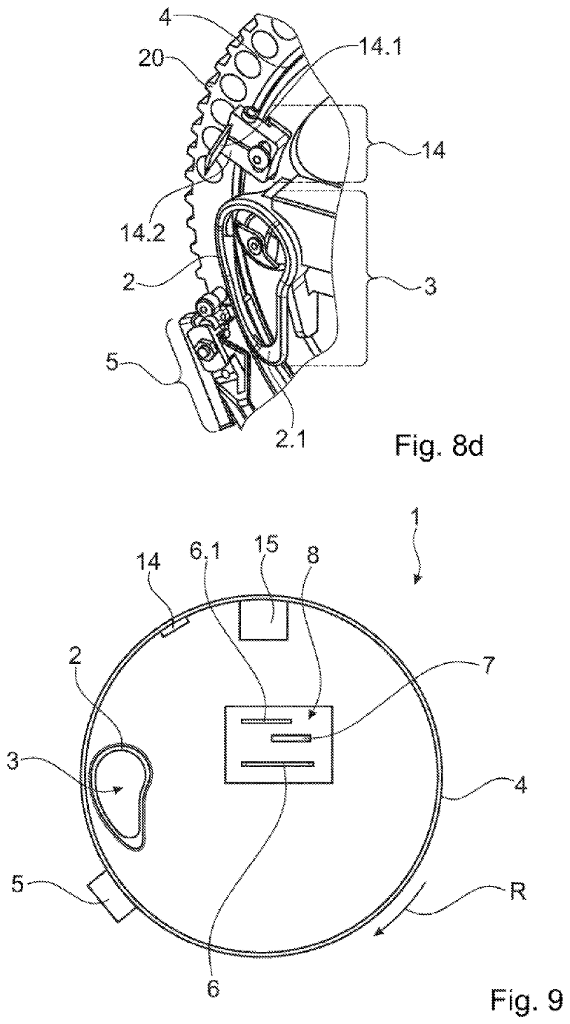

In the present example, the latch arm 10.10 is also spring-loaded with a restoring force via a damper 10.5 with a stop 10.9. Due to the suspension, it is possible that the thread does not break during the envelope. The spring acts directly on the thread end of the driver and ensures a certain play of the thread end (see also spring at the driver in FIG. 8). The thread is thus guided tightly and loosely at the same time, allows enough play for guiding and knotting without breaking.

In this particular embodiment, the holding device is configured to exert a force on the thread loop of between 5 and 25 N, preferably between 7.5 and 15 N, preferably substantially 10 N.

At FIG. 4b the holding device of FIG. 4a is shown, where the latch has been released, i.e. the latch arm is released. In this state, the loop previously held by the holding teeth 10.1, 1.2 is released and transferred to further process control. The spring 10.10 is released and the stop 10.9 is released. The release has taken place via a rotation of the latch lever 10.6 around the latch pin 10.3. In operation, the holding device 10 configured in this way would immediately return to the state shown in FIG. 4a to be ready to receive a new thread loop. In this example, a pneumatic cylinder can be used to return the latch lever to its original position. Depending on the settings of the spring strength and the desired force to be exerted on the thread loop of between 5 and 25 N, preferably of between 7.5 and 15 N, preferably of essentially 10 N, the stop 10.9 must also be pressed against the spring force of the relaxed spring 10.10.

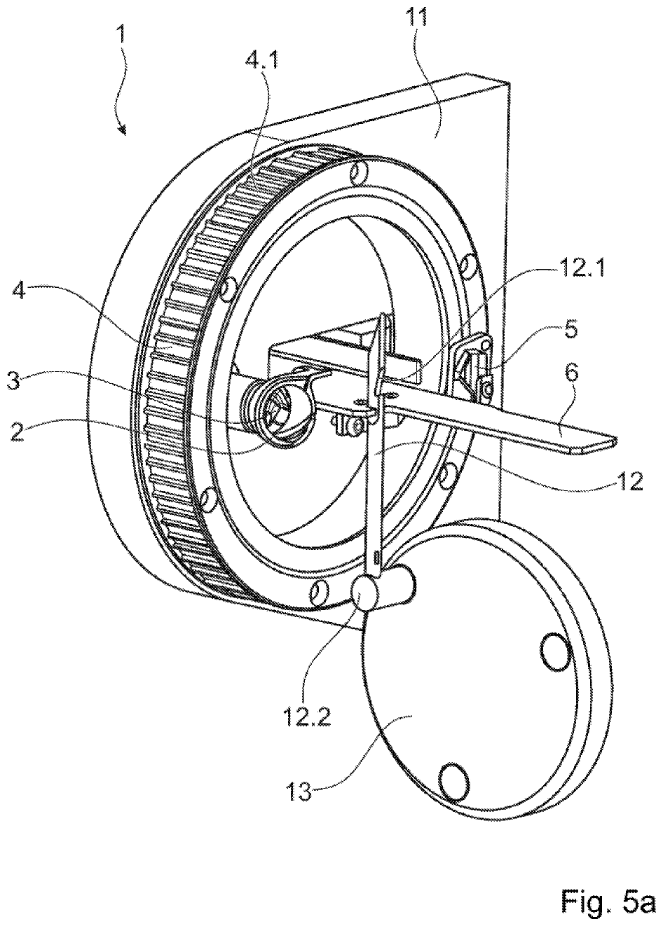

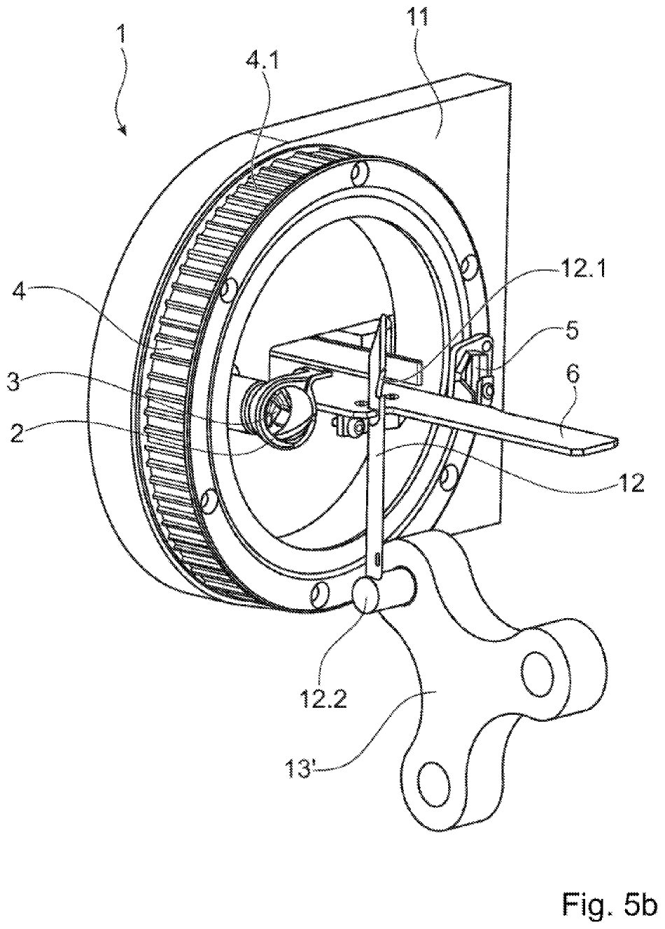

FIG. 5 shows a further advantageous embodiment of a device 1 according to the invention. The device 1 comprises an orbital path 4, which can be driven by means of a toothing 4.1 via a toothed belt. A drive control is provided, which is designed in such a way that a speed of the orbital path 4, which is mounted on the machine frame 11 by means of a ring bearing, can be continuously adjusted.

In particular, said drive control is configured such that the orbital path is capable of traveling at a first speed in a first range and at a second speed in a second range. A driver 5 rigidly connected in the direction of rotation R (clockwise) of the orbital path is carried along.

A tape guide plate 6 extends through the device, through which a ribbon-shaped material enters the effective area of the driver 5 and a loop is formed around the ribbon-shaped substrate by the path of the driver 5 around a holding device 5 and the knotter assembly 3. In order to guide the loop ends to a knotter assembly 3 in a correct orientation, a knotter eyelet 2 is provided, which is radially beveled, and is designed to be aligned in the direction of rotation.

The device further comprises a knotter hook 12 driven by means of a drive wheel 13. The pin-shaped knotter hook 12 includes a hook end 12.1 and a drive end 12.2 connected to and supported by the drive wheel 13. In operation, the hook end 12.2 engages the loop and, next to the plane of rotation of the orbital path, opens it normally to it. For this purpose, the drive pulley 13 is arranged at right angles to the orbital path 4 and driven correspondingly normally to it.

In a particular embodiment, the knotter hook 12 can correctly feed the loop ends to the knotter assembly and/or facilitate the removal of the knotted loop by moving it, after the thread end has been cut, out of the effective region in the direction of rotation of the drive wheel 13 by means of the hook end 12. Lead out.

The drive wheel 13 of the knotter hook 12 is driven so that its movement and speed is synchronous with the driver 5. In other words, the drive wheel can be designed, for example, to complete one full revolution in the time it takes to move the driver once around the entire orbital path.

In a further additional or alternative embodiment, the knotter hook 12 can prevent the thread end from entering the effective region of the cutting element too early during knotting due to the pull of the driver. In this example, this can be facilitated by the knotter hook 12 by pulling on the loop, thus keeping the unfinished knotted loop end away from the cutting element. In this embodiment, the knotter hook 12 would preferably be synchronously clocked with the knotter assembly 3 via the drive wheel 13 so that it completes one revolution in the time it takes the knotter assembly 3 to form a knot, i.e. between 1 and 2 revolutions.

FIG. 5b shows a further advantageous embodiment of the device according to FIG. 5. In order to reduce the overall moving mass, it was surprisingly found that with a drive wheel 13′ with preferably three symmetrically arranged outriggers, a mass saving can be achieved with simultaneous high controllability and quietness of operation. Four- or multi-arm drive wheels would also be conceivable.

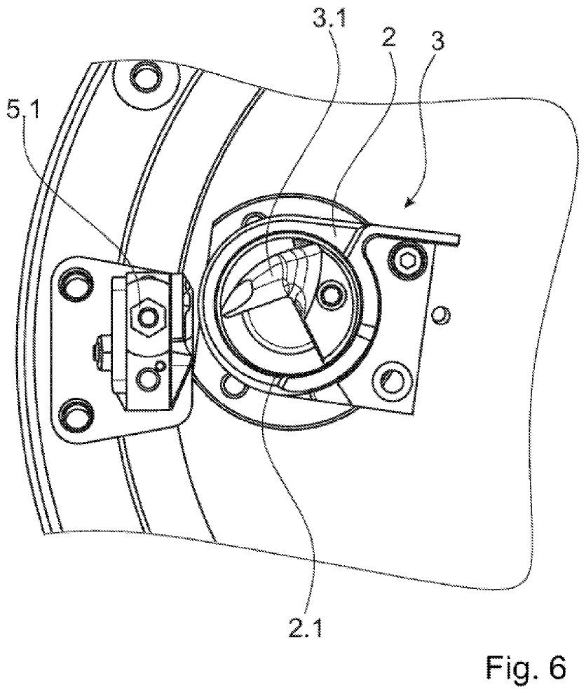

In FIG. 6, a detail is shown of a device as described in the figure descriptions above, which shows a suitable knotter assembly 3 and a driver 5.

The driver 5 shown is rigidly connected to an orbital path 4 and, for illustrative purposes, is located directly adjacent to a knotter assembly 3. The driver can be in this position, for example, at the start of entry into the knotting region. The loop ends of the thread end are not shown for the sake of simplicity.

The knotter assembly 3 is disposed within a knotter eyelet 2 and includes a knotting mandrel 3.1 configured to rotate counterclockwise within the knotter eyelet 2. In the process, the knotting mandrel 3.1 performs a dipping movement with a tip end running towards the thread loops and engages in loop ends held by the driver and holding device. The pressure of the loop ends, the rotation of the knotting mandrel and a guide over the knotter eyelet form a knot in a 1- to 2-fold, preferably one-and-a-half-fold rotation of the knotting mandrel.

The knotter eyelet 2 comprises a guide geometry 2.1 which is aligned towards the outside, i.e. in the radial direction. In operation, a loop end is initially guided to the guide geometry 2.1 and, as it continues along the path of the driver 5, is guided into the effective region of the knotting mandrel 3.1, which rotates in the opposite direction to the orbital path of the driver 5.

In the present example, the driver 5 has a driver gripper 5.1 provided with a restoring force, through which the loop is guided into the knot with sufficient play.

Finally, the second loop end enters the effective region of the knotting mandrel 3.1 and is knotted to the first loop end.

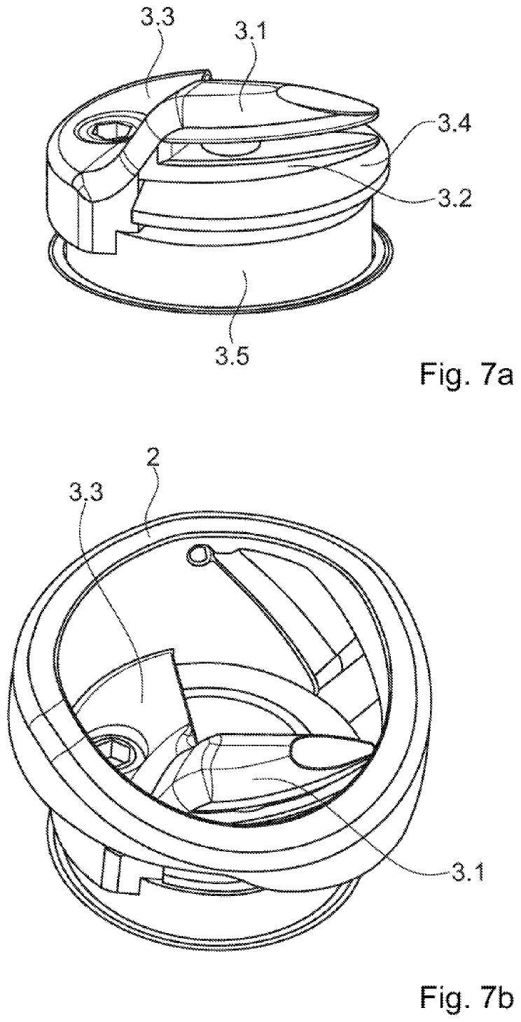

FIG. 7a shows a schematic side view of the knotter assembly of FIG. 6 without the knotter eyelet. The knotter assembly essentially comprises a replaceable knotting mandrel 3.1, which is detachably and rigidly connected to a rotatably mounted drive pulley 3.5 via a mandrel foot 3.3. Overall, the knotter assembly 3 shown has a multi-part design. The knotting mandrel 3.1 includes a first mandrel extension 3.1 and a parallel aligned second mandrel extension 3.2, as well as a thread guide aperture 3.4 also aligned parallel to the knotting mandrel extensions. In operation, the two-part mandrel shape and the thread guide aperture 3.4 ensure that the knotted thread loops can be discharged from the knotter assembly after knotting.

As can be seen in FIG. 7b, the laterally beveled and radially aligned knotter eyelet 2 has a corresponding recess.

Suitable for the device according to the invention is a driver rigidly connected to the orbital path in the direction of rotation. The driver can, for example, be detachably connected to the orbital path by means of a clamping screw or a plurality of clamping screws extending through the entire profile of the orbital path. For this purpose, pre-drilled recesses can be, for example, provided in the orbital path.

A first spring element may likewise be provided on the driver, spanning the entire profile thickness of the orbital path, and connecting a rear element of the driver to a front element of the driver. In the present example, the front element of the driver would be the element that guides the thread end into the effective region of the holding device and the knotting assembly. This allows the first spring element to create an effective connection between springs arranged on the rear side and a thread clamp provided on the front side. In operation, this would result in the thread clamp allowing some play for the thread end due to the spring action. Together, for example, with the spring-loaded holding device described above, this can facilitate knotting by the knotting assembly, prevent breaking of a thread end and enable higher process speeds.

FIG. 8a illustrates another aspect, respectively a particular embodiment, of the present invention. The device shown is suitable for placing a thread end around a ribbon-shaped substrate, for example a cotton tape for a tampon, and knotting two thread sections of this thread end so that a loop can be formed. When this cotton tape is rolled up, wound, and/or folded, these thread sections of the thread end can protrude from a resulting tampon and serve as a retrieval thread. Thus, when the end of the thread is knotted, a loop is formed around a ribbon-shaped substrate. The thread end (not shown) is unwound from a driver 5. The driver 5 is rigidly connected to a toothed rotating wheel 20. This rotating wheel 20 can be driven, for example, by a drive belt, or a drive wheel (not shown), and is preferably respectively acceleratable or deceleratable. The rotating wheel 20 is mounted on an orbital path 4 so that the driver 5 is able to perform a circular movement. In the process, the driver guides the thread end past various processing units.

During a complete rotation around the orbital path 4, the thread is wrapped once around a substrate tape, e.g. cotton tape, which is conveyed centrally into the effective region of the driver. The thread end is first placed around a pickup, which initially holds the thread end and the resulting loop open. A further thread section of the thread end is placed on a deflection fin.

The open thread end is then passed to a knotter assembly 3, where two thread sections of the thread end are knotted together. A guide geometry 2.1 formed on a knotter eyelet ensures that the free thread piece is correctly guided to the thread piece to be knotted. The loop created in this way is fed away in the normal to the image plane together with the cotton tape. The pickup 15 allows the loop created to slide down onto the substrate tape in a controlled manner by releasing the thread section held in a pickup hook 15.1 by lowering a pickup lever 15.4. For this purpose, the pickup lever 15.4 is pivotably mounted in a joint 15.2. A pickup spring 15.3 exerts a restoring force on the pickup hook 15.1. This is overcome by the knotting process of the knotter assembly 3 and corresponding traction on the thread end. When the pickup lever 15.4 is in its maximum lowered position, it releases the thread section, whereupon the restoring force returns it to its angled initial position.

After knotting, the end of the thread is moved past the cutting blade 14, which cuts off the loop. The driver 5 starts a new revolution on the orbital path 4 with a new thread end. The aforementioned elements are attached to a machine frame 11, through which centrally provided recesses allow a tape inlet for the substrate tape.

The substrate tape is guided horizontally by a tape guide plate 6. In addition to the tape guide plate, a stop plate 6.1 is formed parallel to it, which prevents the pickup hook from interfering with the belt conveyance of the ribbon-shaped substrate.

A deflection fin 7 guides the end of the thread from the opposite side close to the cotton tape and enables the loop created to slide on smoothly through its end tapering in the direction of conveyance of the cotton tape, i.e. towards the plane of the observer. In addition, the deflection fin exerts the necessary resistance for the knotter assembly to knot the thread ends by means of a notch. After the loop is finally knotted and a knotter hook (see FIG. 5a or 5b) guides the created loop away with the substrate tape, the deflection fin 7 guides the loop onto the ribbon-shaped substrate through its tapering shape. A bulbous shape following the notch also ensures the necessary pulling resistance to release the loop from the knotter eyelet 2

With the device shown and the resulting gentle deposition of the thread loop onto the cotton tape, a high process speed with high reliability can be achieved. This device is equally capable of driving a corresponding acceleration and variable speed of the driver 5, as described in the embodiment of FIGS. 1 to 7 above.

FIG. 8b shows a section of the pickup 15 at the upper end of the orbital path. The pickup 15 is positioned so that it makes an angle of not more than 95 and not less than 85° degrees with respect to the plane of the tape guide plate (not shown in this figure) and thus to the ribbon-shaped substrate. Here, the angle is viewed from the center of the end face of the pick-up hook 15.1. The pickup hook 15.1 is substantially hook-shaped at the end of a pickup lever 15.4 and is suitable for receiving a thread section of the thread end (not shown). The pickup lever 15.4 is pivoted about a joint 15.2 and is shown angled in FIG. 8b. A pickup spring is operatively connected between the pickup lever 15.4 and the machine frame 11 so that a restoring force acts on the pickup lever 15.4. The pickup 15 is bolted to the machine frame 11 by a flange 22 with a screw 21. An orbital path leads radially around the pickup 15. In FIG. 8b, grooves 23 and teeth 24 can be seen, which can be brought into operative connection with a drive belt or belt, for example, a caterpillar belt.

In FIG. 8c, the pickup lever 15.4 shown in FIG. 8b is shown in an angled position, where the pickup hook 15.1 releases the previously held thread section and allows it to slide onto a substrate tape. The pickup spring 15.3 is fully extended and exerts the full restoring force on the pickup lever. When the thread path has left the pickup hook, the pickup lever moves back to the angled position of FIG. 8b, ready to pick up a new piece of thread.

FIG. 8d shows another area of the embodiment of FIG. 8a in detail. Shown are a knotter assembly 3, a driver 5, and a cutting blade 14. The driver 5 is designed, in operation, to unwind a thread end, forming it into a loop as it travels along the orbital path of the device. Two thread sections are guided towards each other in the knotter assembly 3 in such a way that they can be knotted into a knot. The knotter assembly 3 is a complex component which is surrounded externally by a knotter eyelet 2. The knotter eyelet 2 has a guide geometry 2.1 on the side facing the direction of rotation, which serves to accommodate the free piece of thread. Above the knotter assembly 3, a cutting blade 14 is provided, which is firmly connected to the machine frame via a cutting blade holder 14.2, which in turn holds a replaceably designed blade 14.1 in the effective region of the driver, so that a loop is cut off from the thread end when the driver passes the cutting blade. The driver is rigidly connected to a rotating wheel, which can, for example, be driven by a belt as already described.

The operation of embodiment 8a-d is shown again schematically in FIG. 9.

The device 1 comprises an orbital path 4 around which a driver 5 is movably mounted. The driver 5 thus performs a circular movement around a path inlet 8 provided substantially centrally in the device 1, which serves to convey a ribbon-shaped substrate substantially perpendicular to the image plane into the effective region of the driver 5.

As it moves along its orbital path 4, the driver carries along a thread end whose thread sections interact with the individual elements of the device 1 arranged along the orbital path. First, a loop is formed around the path inlet 8 with the thread end. Two thread sections of the thread end are guided together in the knotter assembly 3 by a knotter eyelet 2 in such a way that a knotting mandrel (not shown) is able to knot them. In this case, a thread section is held by a pickup 15, which is arranged substantially at a right angle above the substrate tape. Subsequently, the thread path is gently lowered onto the substrate tape by the pickup 15 against a restoring force. A stop plate 6.1 may be provided above the ribbon-shaped substrate so that the pickup 15 does not touch it, and so could interfere with the running of the ribbon-shaped substrate. The ribbon-shaped substrate is guided at the path inlet 8 by a guide plate 6.

A cutting blade 14 fixed on the orbital path 4 cuts off the generated loop, which is discharged together with the substrate tape, in particular, for example, by a knotter hook 12 as shown in FIG. 5a or 5b. The loop formed is released from the knotter eyelet of the knotter assembly, and slides over a tapered deflection fin 7 onto the ribbon-shaped substrate at the end face. As the ribbon-shaped substrate is conveyed away, the loop is pulled along with it and can protrude as a retrieval thread in a tampon produced by means of winding, folding and/or pressing.

The present invention provides a device for knotting loose thread ends for the production of tampons with retrieval threads, which is safe in operation as well as easy to maintain and capable of providing continuous high volume tampon production.

### LIST OF REFERENCE NUMBERS

1 Device for knotting a thread end
2 Knotter eyelet
2.1 Guide geometry
3 Knotter assembly
3.1 Knotting mandrel
3.2 Second mandrel extension
3.4 Thread guide aperture
3.5 Rotatably mounted drive pulley
4 Orbital path

### 5 Driver

5.1 Driver gripper
6 Guide plate
6.1 Stop plate

### 7 Deflection fin

8 Path inlet

### 9.2 First pin

### 9.1 Second pin

10 Holding device
10.1 First holding teeth
10.2 Second holding teeth

### 10.3 Latch pin

10.4 Locking plate

### 10.5 Damper

### 10.6 Latch arm

### 10.9 Stop

### 10.10 Spring

11 Machine frame
12 Knotter hook

### 12.1 Hook end

### 12.2 Drive end

13 Drive wheel
14 Cutting blade

### 14.1 Blade

14.2 Cutting blade holder

### 15 Pickup

15.1 Pickup hook

### 15.2 Joint

15.3 Pickup spring
15.4 Pickup levers
20 Rotating wheel

### 23 Groove

### 24 Tooth

### 30 Thread end

B1 Acceleration region
B2 Second acceleration region

### M Center

V Knotting region
R Rotation direction
U Turnover region

1. A device (1) for knotting a thread end so that a loop is formed, in particular a thread end of a retrieval thread for tampons, comprising:
a. a knotter assembly (3) for knotting two thread sections of the thread end;
b. a driver (5) for feeding the thread sections to the knotter assembly (3);
c. an orbital path (4) along which the driver is guided around the knotter assembly (3),
wherein the orbital path has a knotting region (V) in which the driver is movable at a first speed, and an envelope region (U) in which the driver is movable at a second speed.

2. The device of claim 1, further comprising a thread feed for continuously feeding a thread end to the driver.

3. The device of claim 1, further comprising a cutting element for severing the loop of the thread end.

4. The device of claim 1, wherein the orbital path is a substantially circular orbital path.

5. The device of claim 1, comprising a path inlet for passing a ribbon-shaped substrate through the effective region of the driver so that the thread end is laid around the ribbon-shaped substrate, in particular so that the thread end is laid around the ribbon-shaped substrate when the driver orbits on the orbital path.

6. The device of claim 1, further comprising a holding device, in particular a holding device designed as a latch, which is designed to exert a restoring force on a loop end of the thread end to be knotted.

7. The device of claim 5, further comprising a guide plate for guiding ribbon-shaped substrate through the active region of the driver.

8. The device of claim 1, further comprising a knot control for checking the knot of two thread sections tied by the knotter assembly (3).

9. The device of claim 1, wherein the orbital path is drivable, in particular comprises a toothing by which the orbital path is drivable by means of a toothed belt so as to be capable of performing an acceleratable rotation about an axis of rotation.

10. The device of claim 1, comprising a direct drive for driving the driver, in particular an electric direct drive.

11. The device of 10 claim 1, further comprising an orbital drive, in particular a continuously acceleratable orbital drive, for driving the driver on the orbital path.

12. The device of claim 1, wherein the knotter assembly comprises a knotting mandrel which is rotatably driven.

13. The device of claim 1, wherein the device is configured to guide the driver through a knotting region at a first speed, wherein the first speed is lower than a second speed at which the driver is guided through the envelope region, in particular wherein the first speed has a maximum speed that is lower than a maximum speed of the second speed by between two and nine times.

14. The device of claim 1, wherein the first speed is between 300 and 600°/s and the second speed is between 500 and 4000°/s.

15. The device of claim 14, wherein the device is configured to guide the driver at a first speed through a knotting region of between 50 and 150 rpm, particularly about 90 rpm, and to guide the driver at a second speed through an envelope region of between 200 rpm and 600 rpm, particularly about 500 rpm.

16. The device of claim 1, further comprising a drive control system for moving the driver in the knotting region at a first speed, and in an envelope region (U) at a second speed, in particular wherein the drive control system is adapted to guide the driver at a first speed through the knotting region so that this leaves the knotting region after the knotter assembly has performed between one and two, in particular substantially one and a half revolutions against the direction of the driver.

17. A method for knotting a thread end, in particular two thread sections of the thread end, so that a loop is formed which forms a retrieval thread for tampons, comprising the steps of:
a. guiding a thread end of a thread by means of a driver movable on an orbital path along an orbital path around a knotter assembly and an envelope element, so that a loop of two thread sections of the thread end is formed between the knotter assembly and the envelope element;
b. knotting the two thread sections by the knotter assembly and untying the loop at the envelope element, and
wherein the orbital path has a knotting region (V) in which the driver is guided at a first speed, and has an envelope region (U) in which the driver is guided at a second speed.

18. The method of claim 15, wherein
a. a feeding of a thread end from a thread on a bobbin on the driver movable over the orbital path occurs via a restoring force, and
b. the releasing of the loop comprises cutting the thread end from the thread.

19. The method of claim 15, wherein the ratio of the first speed to the second speed is between 1:2 and 1:6.

20. The method of claim 16, wherein the knotting region (V) includes an angle of between 5 and 45 degrees of the orbital path, preferably including between 5 and 30 degrees, preferably including 15 degrees.

21. The method of any claim 15, wherein the untying of the loop on the envelope element provides for a releasing of a latch configured to exert a releasable restoring force in the direction of the driver and/or knotter assembly.

22. The method of claim 15, further comprising the steps of:
a. providing a device (1) according to claim 1 for knotting a thread end so that a loop is formed;
b. passing a ribbon-shaped substrate through an effective region of the driver so that the thread end is laid around the ribbon-shaped substrate, in particular so that the thread end is laid around the ribbon-shaped substrate when the driver orbits on the orbital path, wherein the passing of the ribbon-shaped substrate is arranged substantially normal to an orbital path of a driver.

23. A device for producing tampons having a proximal retrieval thread comprising two thread ends linked together, the device comprising:
a. the device for knotting two thread ends according to claim 1,
b. a conveying device for conveying ribbon-shaped substrate into an effective region of the device for knotting two thread ends, in particular wherein the conveying device is arranged substantially perpendicular to an orbital path of a driver for feeding the two threads to a knotter assembly, and
wherein the conveying device for conveying the ribbon-shaped substrate conveys into the effective region of the device for knotting two thread ends in such a way that, during an orbital movement of a driver along an orbital path, the thread is laid around the ribbon-shaped substrate substantially transversely to the longitudinal axis thereof.

24. A device (1) for knotting a thread end so that a loop is formed around a ribbon-shaped substrate, in particular a thread end of a retrieval thread for tampons, comprising:
a. a knotter assembly (3) for knotting two thread sections of the thread end;
b. a driver (5) for feeding the thread sections to the knotter assembly (3), wherein the driver is rotatable along an orbital path (4) around a path inlet (8) for passing a ribbon-shaped substrate, in particular rotatable on a circular orbital path (4), so that a loop is formed,
a pickup (15) arranged substantially normal to the path inlet (8) is provided to receive a thread section from the driver and deposit it onto the ribbon-shaped substrate.

25. The device of claim 24, wherein the pickup (15) is substantially metal.

26. The device of claim 24, comprising a pickup spring (15.3) which exerts a restoring force on the pickup (15).
